(19) United States
(12) Patent Application Publication
(10) Pub. No.: US 2015/0196437 A1
(43) Pub. Date: Jul. 16, 2015

(54) WIPE AND OINTMENT POUCH

(71) Applicant: Svetlana Gerasimova-Culp, Chicago, IL (US)

(72) Inventor: Svetlana Gerasimova-Culp, Chicago, IL (US)

(21) Appl. No.: 14/155,193

(22) Filed: Jan. 14, 2014

(51) Int. Cl. A61F 13/551

(52) U.S. Cl. CPC A61F 13/5519

(57) ABSTRACT

A wipe pouch may include a housing having a front side, a rear side and a main cavity. In certain embodiments the housing may have a secondary cavity. The main cavity and the secondary cavity may open out to the rear side of the housing. A lid may be attached to the rear side of the housing. A fastening component layer may cover the lid. In a standalone embodiment, the fastening component layer may be covered by a fastening component cover. In certain embodiments, the wipe pouch may be attached to a diaper as a built in component. In certain embodiments, at least one wipe may be enclosed in the main cavity and an ointment may be enclosed in the secondary cavity.

### BACKGROUND OF THE INVENTION

The present invention relates to diapers and, more particularly, to a wipe and ointment pouch that attaches to a diaper

Currently, baby wipes are difficult to keep in close proximity in order to have them at a ready access to clean a baby when the baby's diaper needs to be changed. The diaper and the baby wipes are treated as separate tools. These items are complimentary, and should be conveniently attached to each other. Currently, baby wipes are carried separately and usually in bulking packaging. Many times while moving in haste, the baby wipes are forgotten and not available when needed.

As can be seen, there is a need for a wipe and ointment pouch that attaches to a diaper.

### SUMMARY OF THE INVENTION

In one aspect of the present invention, a wipe pouch comprises: a housing having a front side, a rear side and a main cavity, wherein the main cavity opens out to the rear side of the housing; a lid attached to the rear side of the housing; and a fastening component layer covering the lid on the housing.

In another aspect of the present invention, a wipe and ointment pouch comprises: a housing having a front side, a rear side, a main cavity and a secondary cavity, wherein the main cavity and the secondary cavity opens out to the rear side of the housing; a lid attached to the rear side of the housing; and a fastening component layer covering the lid on the housing.

In another aspect of the present invention, diaper with a wipe and ointment pouch comprises: a diaper; a housing having a front side, a rear side, a main cavity and a secondary cavity, wherein the main cavity and the secondary cavity opens out to the rear side of the housing; a lid attached to the rear side of the housing; and a fastening component layer covering the lid on the housing, wherein the fastening component layer attaches to the diaper.

In another aspect of the present invention, a method of ready access wipe and ointment for changing a diaper of a baby comprises: prefilling a main cavity of a wipe pouch with at least one wipe; attaching a fastening component layer of the wipe pouch to the diaper of the baby, wherein the wipe pouch comprises a housing having a front side, a rear side and a main cavity, wherein the main cavity and the secondary cavity opens out to the rear side of the housing; a lid attached to the rear side of the housing; and the fastening component layer covering the lid on the housing; waiting until the diaper of the baby is full; opening the wipe pouch to access the wipes in the main cavity; and using the wipes on the baby while in the process of changing the diaper of the baby.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a perspective view of an exemplary embodiment of the present invention;

FIG. 3 is a rear view of an exemplary embodiment of the present invention;

FIG. 4 is an exploded view of an exemplary embodiment of the present invention, illustrating the placement of the pouch onto a diaper;

FIG. 5 is a section view of an exemplary embodiment of the present invention, taken along line 5-5 in FIG. 2; and

FIG. 6 is a section view of an exemplary embodiment of the present invention, taken along line 6-6 in FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a wipe pouch that may include a housing having a front side, a rear side and a main cavity. In certain embodiments the housing may have a secondary cavity. The main cavity and the secondary cavity may open out to the rear side of the housing. A lid may be attached to the rear side of the housing. A fastening component layer may cover the lid. In a standalone embodiment, the fastening component layer may be covered by a fastening component cover. In certain embodiments, the wipe pouch may be attached to a diaper as a built in component. In certain embodiments, at least one wipe may be enclosed in the main cavity and an ointment may be enclosed in the secondary cavity.

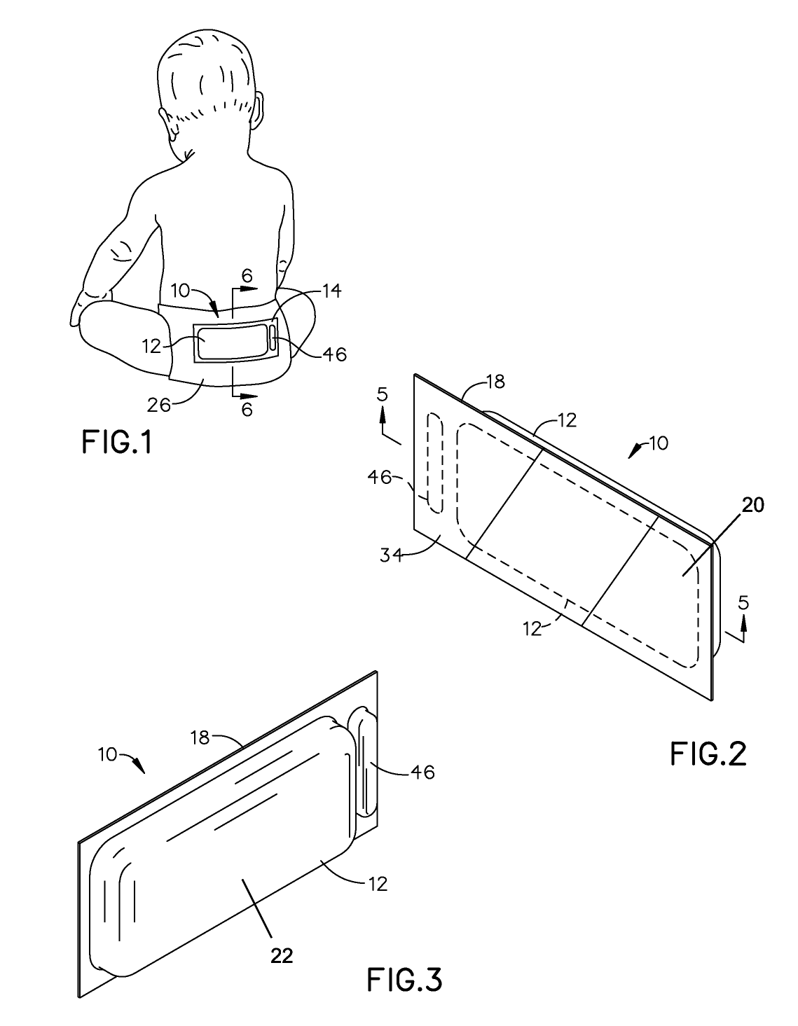

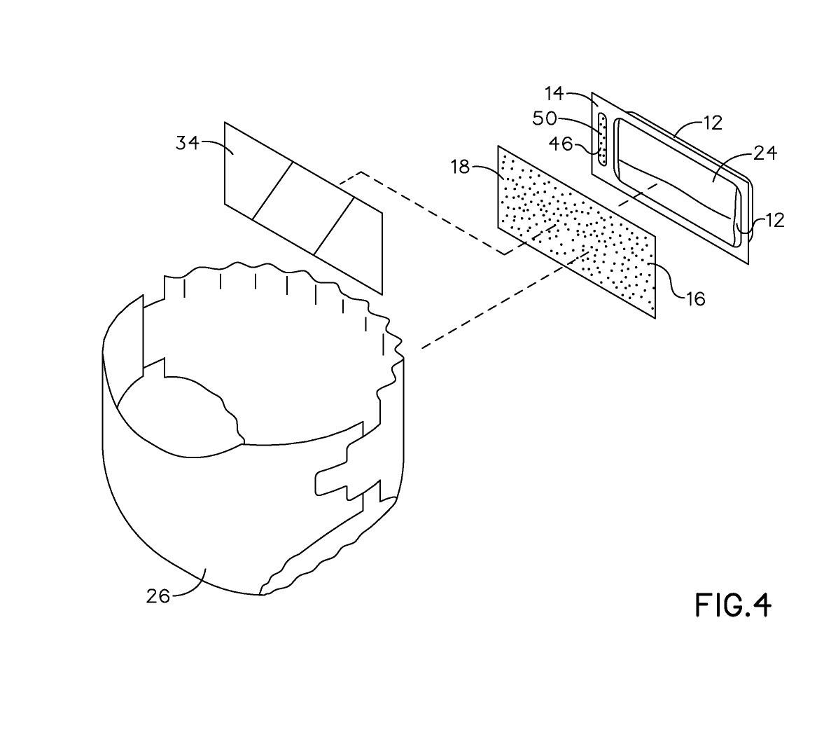

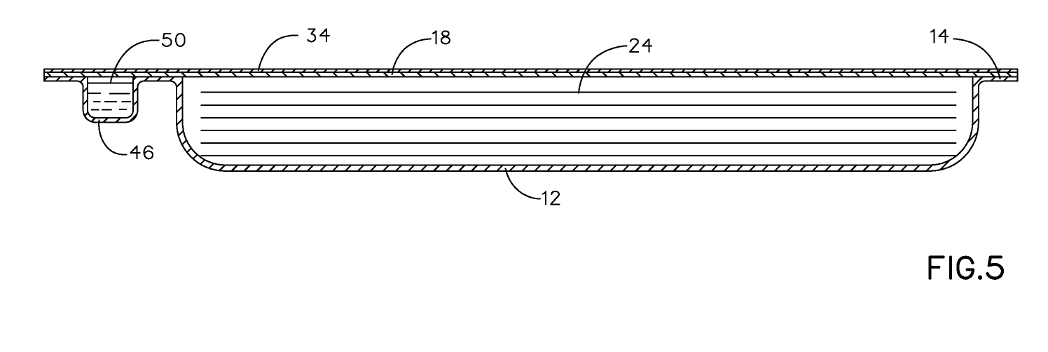

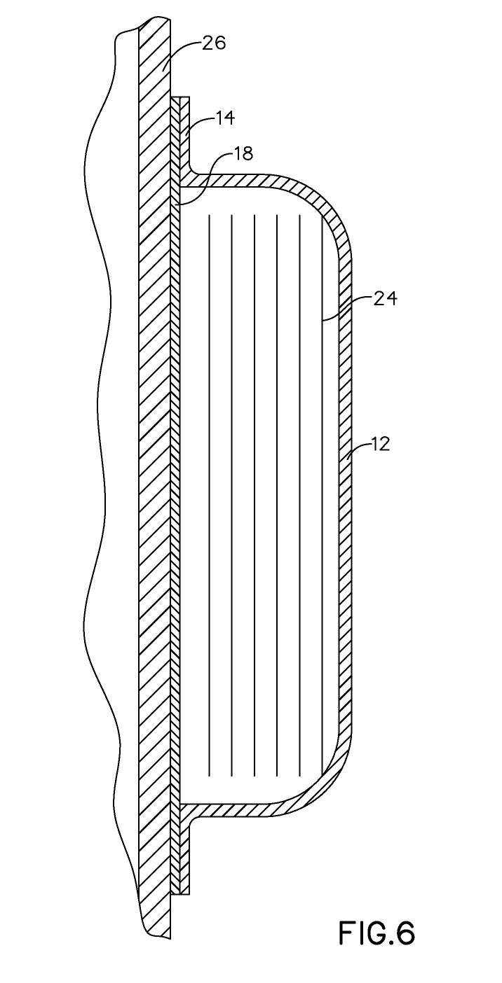

As is illustrated in FIGS. 1 through 5, a wipe pouch 10 may include a housing 14. The housing 14 may have front side 22, a rear side 20 and a main cavity 12. In certain embodiments, the housing 14 may have a secondary cavity 46. The main cavity 12 and the secondary cavity 46 may open out to the rear side 20 of the housing 14. A lid 18 may attach to the rear side 20 of the housing 14. A fastening component layer 16 may cover the lid 18 on the housing 14. In certain embodiments, a fastening component cover 34 may be removably attached to the fastening component layer 16.

In certain embodiments, the wipe pouch 10 may be added to a diaper 26. In certain embodiments, the wipe pouch 10 may be built into a diaper 26. In certain embodiments, the wipe pouch 10 may include access for at least one wipe 24 that may be enclosed in the main cavity 12 of the housing 14. In certain embodiments, the wipe pouch 10 may include access for an ointment 50 that may be enclosed in the secondary cavity 46 in the housing 14 as well. The fastening component layer 16 may be an adhesive or the like.

In certain embodiments, the wipe pouch 10 may be a standalone device. In certain embodiments, the wipe pouch 10 may be attached to a diaper 26 while in manufacturing. When using wipe pouch 10 in a standalone situation, an individual may attach the wipe pouch 10 to any brand of diaper 26. This flexibility allows the individual to purchase the wipe pouch 10 and the diapers 26 separately, but use them together. The ability to attach the wipe pouch 10 to the diaper 26 may eliminate the need to carry unnecessary amounts of baby wipes 24. When attached to the diaper 26, the wipes 24 may not be forgotten at home when the baby needs a change of diaper 26.

A method of using the wipe pouch 10 may include the following. An individual may have the main cavity 12 of the wipe pouch 10 prefilled with at least one wipe 24. In certain embodiments, the individual may also have the secondary cavity 46 prefilled with ointment 50. The fastening component layer 16 of the wipe pouch 10 may be attached to the diaper 26 of the baby. The individual may wait until the diaper 26 of the baby is full. When ready to change the diaper 26 of the baby, the individual may open the wipe pouch 10 to access the at least one wipe 24 from the main cavity 12. The individual may then use the wipes 24 on the baby while in the process of changing the diaper 26 of the baby. In certain embodiments when the secondary cavity 46 may be prefilled with ointment 50, and the ointment 50 is needed, the individual may access the ointment 50 from the secondary cavity 46 to apply to the baby. When a new diaper 26 may be put on the baby, a diaper 26 with a wipe pouch 10 built on the diaper 26 may be used. In other embodiments, a standard diaper 26 may be used, and a wipe pouch 10 may be attached to the diaper 26 by way of the fastening component layer 16. The stand alone wipe pouch 10 may have a fastening component cover 34 attached to the fastening component layer 16 in order to keep the fastening component layer 16 from fastening when the fastening component layer 16 may not be near the diaper 26. The used wipes 24 may be thrown away with the dirty diaper 26. In certain embodiments, the diaper 26 and wipe pouch 10 may be used for adults that have to wear diapers 26.

It should be understood, of course, that the foregoing relates to exemplary embodiments of the invention and that modifications may be made without departing from the spirit and scope of the invention as set forth in the following claims.

1. A wipe pouch comprising:
a housing having a front side, a rear side and a main cavity, wherein the main cavity opens out to the rear side of the housing;
a lid attached to the rear side of the housing; and
a fastening component layer covering the lid on the housing.

2. The wipe pouch of claim 1, further comprising at least one wipe enclosed in the main cavity.

3. The wipe pouch of claim 1, further comprising a fastening component cover removably attached to the fastening component layer.

4. The wipe pouch of claim 3, wherein the fastening component layer is an adhesive.

5. The wipe pouch of claim 1, further comprising
a secondary cavity, wherein the secondary cavity opens out to the rear side of the housing.

6. The wipe and ointment pouch of claim 5, further comprising at least one wipe enclosed in the main cavity and an ointment enclosed in the secondary cavity.

7. The wipe and ointment pouch of claim 5, further comprising a fastening component cover removably attached to the fastening component layer.

8. The wipe and ointment pouch of claim 7, wherein the fastening component layer is an adhesive.

9. A diaper with a wipe and ointment pouch comprising:
a diaper;
a housing having a front side, a rear side, a main cavity and a secondary cavity, wherein the main cavity and the secondary cavity opens out to the rear side of the housing;
a lid attached to the rear side of the housing; and
a fastening component layer covering the lid on the housing, wherein the fastening component layer attaches to the diaper.

10. The diaper with a wipe and ointment pouch of claim 7, wherein the fastening component layer is an adhesive.

11. A method of ready access wipe and ointment for changing a diaper of a baby comprising:
prefilling a main cavity of a wipe pouch with at least one wipe;
attaching a fastening component layer of the wipe pouch to the diaper of the baby, wherein the wipe pouch comprises a housing having a front side, a rear side and a main cavity, wherein the main cavity and the secondary cavity opens out to the rear side of the housing; a lid attached to the rear side of the housing; and the fastening component layer covering the lid on the housing;
waiting until the diaper of the baby is full;
opening the wipe pouch to access the wipes in the main cavity; and
using the wipes on the baby while in the process of changing the diaper of the baby.

12. The method of claim 11, further comprising the step of prefilling the secondary cavity with an ointment.

13. The method of claim 12, further comprising the step of using the ointment during the process of changing the diaper of the baby.
